(19) United States
(12) Patent Application Publication
(10) Pub. No.: US 2024/0257935 A1
(43) Pub. Date: Aug. 1, 2024

(54) ROBOTIC PRESCRIPTION FILLING SYSTEM

(71) Applicant: CareFusion 303, Inc., San Diego, CA (US)

(72) Inventors: Dennis A. TRIBBLE, Ormond Beach, FL (US); Doina J. DUMITRU, The Woodlands, TX (US)

(21) Appl. No.: 18/561,652

(22) PCT Filed: May 19, 2022
(86) PCT No.: PCT/US2022/030127

§ 371 Date: Nov. 16, 2023

Related U.S. Application Data
(60) Provisional application No. 63/209,577, filed on Jun. 11, 2021.

(51) Int. Cl. G16H 20/13

(52) U.S. Cl. CPC G16H 20/13

(57) ABSTRACT

Unattended robotic pharmacy systems are provided that include an enclosure defining a controlled access space and a prescription filling unit disposed within the controlled access space, the prescription filling unit configured to package and label a patient prescription of medication obtained from medication storage within the prescription filling unit. A finished prescription unit is disposed within the controlled access space and coupled to the prescription filling unit, the finished prescription unit configured to store the packaged and labeled patient prescription obtained from the prescription filling unit. An interface kiosk is coupled to the enclosure and disposed adjacent the finished prescription unit, the interface kiosk configured to receive input from a user and to dispense a finished prescription obtained from the finished prescription unit to the user.

### CROSS REFERENCE TO RELATED APPLICATIONS

This is a U.S. national stage application of International Patent Application No. PCT/US2022/030127, entitled “ROBOTIC PRESCRIPTION FILLING SYSTEM,” filed May 19, 2022, which claims the benefit of and priority to U.S. provisional patent application 63/209,577, entitled “ROBOTIC PRESCRIPTION FILLING SYSTEM,” filed Jun. 11, 2021, all of which are incorporated herein by reference in their entirety.

### TECHNICAL FIELD

The present disclosure generally relates to prescription filling systems, in particular remote robotic prescription filling systems for unattended use.

### BACKGROUND

A prescription is an instruction from a prescriber, typically, but not always, a physician, to a pharmacist to prepare and label a supply of medication for a patient for self-administration. Such prescriptions are typically sold to the patient in a retail drug store or similar establishment. In a typical pharmacy, a pharmacist or staff member under the direct guidance of a pharmacist, manually retrieves medications from various storage units, packages the prescription, and labels the prescription for dispensing to a patient. If the individual is not a pharmacist, a pharmacist is required to inspect and approve the packaged prescription.

In some cases, a typical prescription filling robotic device maintains a supply of oral solid medications, prescription vials, and labels, and, upon input from a Pharmacy Information System, fills and labels a prescription for local inspection by a pharmacist, thus requiring attended use. The pharmacist then verifies accurate filling and labeling, and either releases the filled prescription to the patient or sets it aside for subsequent pickup. However, these devices cannot handle liquid medications, medications intended to be reconstituted (e.g., liquified) immediately before release, or semi-solid medications, such as creams, ointments or lotions.

In addition, personal inspection of the finished prescription by a pharmacist requires that a pharmacist be physically present or virtually present to observe and certify the filling process. In many jurisdictions, the part of the pharmacy in which prescriptions are filled (e.g., the actual pharmacy as opposed to the rest of a retail establishment) may not be open in the absence of a pharmacist. Some jurisdictions have permitted pharmacist presence to be rendered remotely over telecommunications and digital connections, but still require that the pharmacist observe the process. These processes typically require that there be a physical person, such as a registered or licensed pharmacy technician, to physically oversee and perform prescription filling with the pharmacist observing and certifying specific steps in the process.

Thus, when a pharmacist is not present, virtually or physically, prescriptions cannot be filled, nor can filled prescriptions be dispensed to patients. This makes it difficult for individuals who work long or unusual hours and may not be able to visit the pharmacy when it is open. It is also difficult for individuals who live in remote areas who may be physically very distant from a pharmacy where there is insufficient business reason to establish a pharmacy near to them, which is related mostly to a lack of appropriate professional individuals to staff such a remote pharmacy.

In some cases, mail order pharmacies are known for handling filling and refilling of non-emergency medications. However, mail order pharmacies cannot respond quickly enough to deliver medications needed immediately, such as pain medications or antibiotics. In other cases, physician-dispensing systems are known, which may be robotic, but which require the physician or office staff to act as surrogates for the pharmacist and to physically verify the labeling and supply the medication to the patient. Further, such physician-dispensing systems are not available except when the patient is in the physician's office.

For these reasons, it is desirable to provide automated prescription filling and dispensing systems for unattended filling of all types of prescriptions without requiring a pharmacist to be physically present in order to make pharmacy services available during off hours and to make pharmacy services available to individuals who are remote from established pharmacies.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a typical prescription filling robotic device.

FIG. 2 depicts another typical prescription filling robotic device.

FIG. 3 depicts yet another typical filling robotic device.

FIG. 4 depicts a schematic top view of a robotic prescription filling and dispensing system, according to aspects of the disclosure.

FIG. 5 depicts a front view of an interface kiosk of the robotic prescription filling and dispensing system of FIG. 4, according to aspects of the disclosure.

FIG. 6 depicts a block diagram illustrating an example computer system with which the robotic prescription filling and dispensing system of FIGS. 4 and 5 can be implemented.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Robotic devices currently exist that are intended for attended use. These devices maintain a supply of oral solid medications, prescription vials, and labels, and, upon input from a Pharmacy Information System, fill and label a prescription for local inspection by a pharmacist who then verifies accurate filling and labeling, and releases the prescription to the patient or sets the prescription aside for subsequent pickup. However, these devices cannot handle liquid medications or medications intended to be reconstituted (e.g., liquefied) immediately before release, nor are they designed to handle semi-solid medications.

There are also box-picker devices that can print and affix a label to a boxed item stored in their inventory. However, these devices cannot prepare prescription supplies for oral medications in vials, nor can they deliver aliquots of liquids for prescription-filling.

There are also IV workflow systems in the marketplace that guide human users through a prescription preparation process (e.g., specifically for IV infusion doses) that contain some data capture and remote verification features. However, these systems require human activity and oversight. What distinguishes this notion is that the process does not involve human activity, and so requires a highly reliable robotic system to perform tasks currently performed by humans with sufficient documentation that it would meet regulatory needs for human inspection.

According to aspects of the disclosure, an unattended robotic prescription filling and dispensing system fills and labels prescriptions from source containers, maintains process metadata and images, presents those images for pharmacist verification (where it may be required by law), stores the prescription for pick-up by the patient, supplies credentials to the patient, and releases the prescription to the patient or family member or caregiver upon presentation of the supplied credentials. For example, a source container may be any of a bulk bottle of oral solid pills, a bulk bottle of liquid, a bulk container of cream or ointment, a unit-of-issue bottle of oral solids, a unit-of-issue box of oral solids, a unit-of-issue bottle of liquid and a unit-of-issue cream or ointment, where unit-of-issue is a manufacturer's or pharmacy's pre-packaged container of a medication in commonly-prescribed quantities.

According to aspects of the disclosure, a robotic prescription filling and dispensing system may maintain supplies of medications in various forms (e.g., bulk pills, boxes of pills in standard quantities, bottles of pills in standard quantities, liquids, and powders for reconstitution), as well as a supply of water for reconstitution. The robotic system may include a label supply to print and apply prescription labels to completed prescriptions.

In aspects of the disclosure, a robotic prescription filling and dispensing system may capture both data and images from the prescription preparation process that can be used by a pharmacist to remotely inspect the finished prescription preparation. For example, the information captured may include photographs of the actual supplies and containers used, National Drug Code (NDC) of the medication used (e.g., acquired from a photograph, a bar code, or the storage system from which it is retrieved), lot number, expiration date and records relating to the temperature and humidity (e.g., where required) at which the retrieved item(s) has/have been stored.

In aspects of the disclosure, a robotic prescription filling and dispensing system may have a refrigeration device for medications requiring refrigerated storage.

In aspects of the disclosure, a robotic prescription filling and dispensing system may use bar code verification and image capture to ensure selection of the appropriate source container.

In aspects of the disclosure, for prescriptions that require the transfer of medication from a source container into a final prescription container, a robotic prescription filling and dispensing system may use an electronic balance to verify that the correct quantity had been transferred. For example, this may be applied to either liquids or oral solids.

In aspects of the disclosure, for prescriptions that required the transfer of oral solid medications, a robotic prescription filling and dispensing system may use a counting mechanism to count out the oral solids and an electronic balance to verify that all medications were captured in the final prescription container. Here, the robotic prescription filling and dispensing system may take an image from the top of the container, thus permitting a remote pharmacist to see the medications in the container. The robotic prescription filling and dispensing system may also maintain a database of images associated with each national drug code (NDC) in order to show the remote pharmacist what the oral solids should look like.

In aspects of the disclosure, for prescriptions that required the transfer of fluids, a robotic prescription filling and dispensing system may maintain a measurement system that uses disposable components to measure and deliver the fluid into the final container. The robotic prescription filling and dispensing system may verify the fluid measurement by an electronic balance.

In aspects of the disclosure, for prescriptions that require only to have a label affixed to a pre-filled container, a robotic prescription filling and dispensing system may select the appropriate medication from its inventory and apply the printed label. These include, but are not limited to, pre-filled bottles of pills, pre-filled boxes of pills, pre-filled bottles of liquids, bottles of topical fluids and tubes of topical creams and ointments (e.g., semi-solids).

In aspects of the disclosure, for prescriptions involving reconstitution of powders for suspension (e.g., oral antibiotics), a robotic prescription filling and dispensing system may store the source container in a labeled, but non-reconstituted form until picked up by the patient, at which time device may reconstitute the powder with an appropriate volume of the appropriate type of water and apply agitation appropriate to ensure complete suspension of the powder.

In aspects of the disclosure, each filled prescription may be set aside in storage until it is checked by a remote pharmacist. Once checking is successfully completed, each filled prescription may be stored for retrieval by the patient and a notification may be sent to the patient to come pick it up. Where state law may permit, such review may be omitted or performed on a statistical basis to verify that the robotics are functioning properly.

In aspects of the disclosure, each source container may be tracked against its expiration date and prompted for removal if expired. Also, a robotic prescription filling and dispensing system may notify a management service when it needs to be replenished, which would prompt a visit to the device.

In aspects of the disclosure, there may be a network of robotic prescription filling and dispensing systems in various locations configured to serve patients for whom there is not a nearby pharmacy run by a management application.

In aspects of the disclosure, as prescriptions are filled and checked by a particular robotic prescription filling and dispensing system, an associated management application may be notified and may, in turn, notify the patient by email, text or telephone call that their prescription is ready for pickup. The management application and/or the robotic prescription filling and dispensing system may supply credentials or the ability for the patient to supply their own credentials, which might include any of a password, a bar code or QR code with access credentials, and information from a credit card associated with the patient's account.

In aspects of the disclosure, the management application may track the inventory in each robotic prescription filling and dispensing system and prompt for refills in sufficient time to permit ongoing use of the robotic prescription filling and dispensing system. Each robotic prescription filling and dispensing system may have a formulary of medications available for prescription filling that may be changed within the management application as the needs of the population being served change. In the case where the nearest robotic system did not have a supply of the medication required, the management system would direct filling to the nearest robotic filling system that contained the medication and/or would prompt for supply of that medication to the nearest (to the patient) robotic device and notify the patient of the delay in filling.

In aspects of the disclosure, a robotic prescription filling and dispensing system may produce boxes of foil-packaged oral-solid medications that are useful especially for relatively small prescription volumes for medications with sensitivity to moisture.

In aspects of the disclosure, a robotic prescription filling and dispensing system may package and photo-inspect medications in compliance packaging in which all the chronic oral solid medications used by a patient are packaged into a strip of sequential pouches, where each pouch contains all the medications for that patient to be taken at a particular time. Such a robotic prescription filling and dispensing system may include a mechanism to store and deliver medications to a packaging system that prepares such strips of pouches and a mechanism to photo-inspect the resulting strips to ensure that they are properly filled.

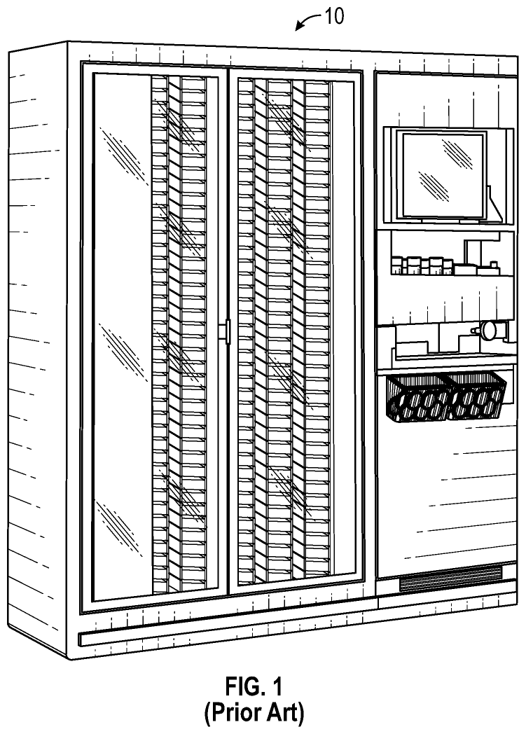

FIGS. 1-3 depict typical robotic prescription filling systems used in manned dispensing procedures. For example, in FIG. 1 a robotic prescription filling system 10 has a storage cabinet with bulk dispensing containers that fills vials with prescribed solid drug portions, prints and applies labels and delivers the uncapped vials for final manned inspection.

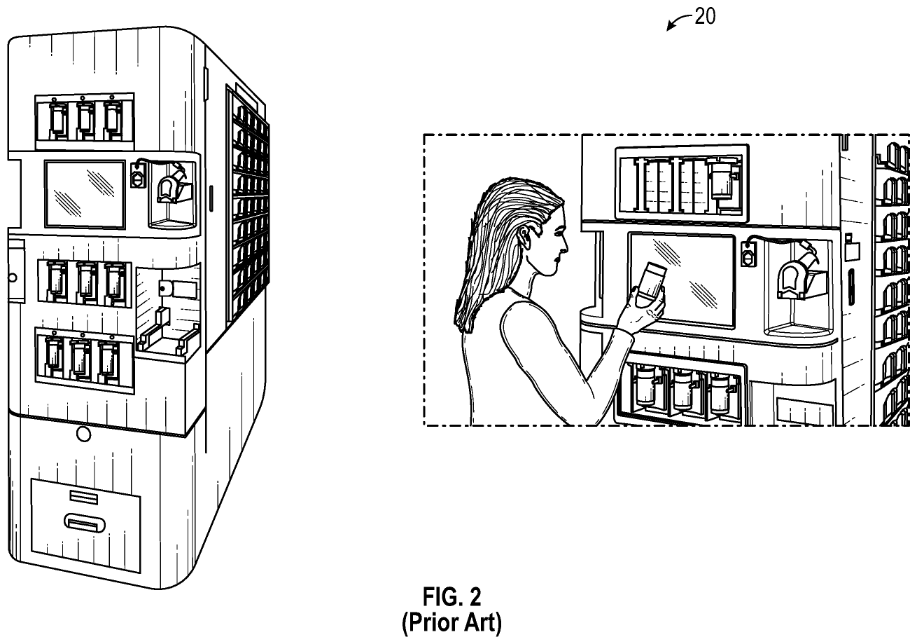

As another example shown in FIG. 2, a robotic prescription filling system 20 similarly has a storage cabinet with bulk dispensing containers that fills vials with prescribed solid drug portions, prints and applies labels and delivers the uncapped vials for inspection, closing and final packaging by a pharmacist or pharmacy staff member.

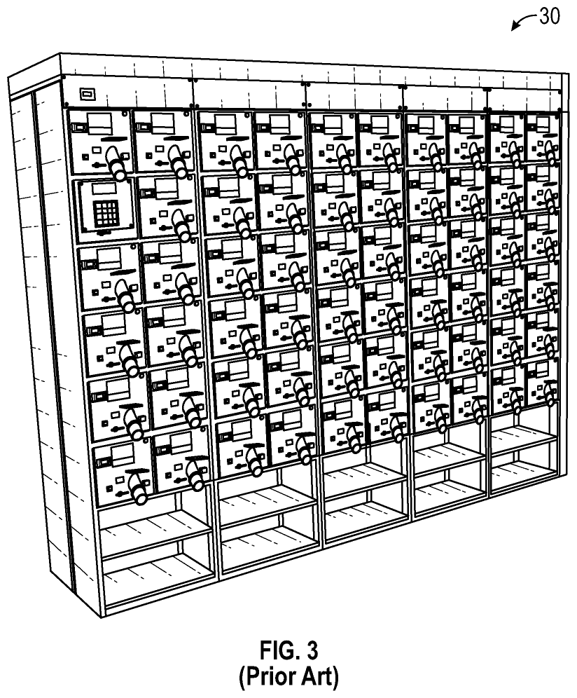

Another robotic prescription filling system 30 is shown in FIG. 3, which has a storage cabinet with bulk dispensing containers and delivery chutes that fills handheld vials with prescribed solid drug portions. Again, a pharmacist or pharmacy staff member is required to hold the vial under the correct chute, inspect the resulting fill and package the vial for dispensing to a patient.

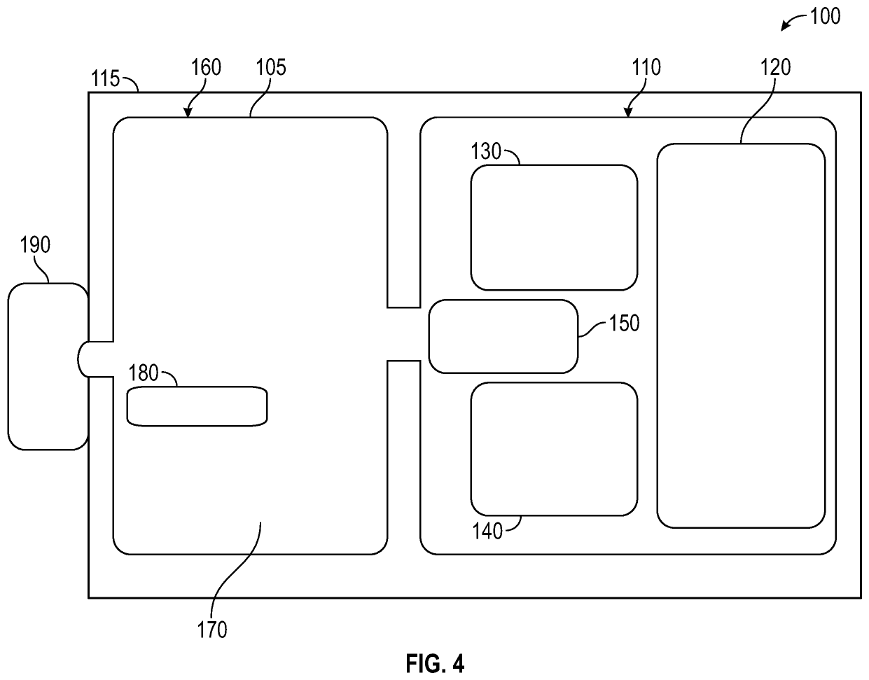

According to aspects of the disclosure, a robotic prescription filling and dispensing system 100 (e.g., robotic pharmacy system) is shown in FIG. 4. Robotic pharmacy system 100 is configured for unattended pharmacy operation, in particular filling and dispensing of prescriptions. For example, robotic pharmacy system 100 may be located in a rural area that is more accessible to a particular patient community not served by the usual manned pharmacy having pharmacists on location to fill and dispense prescriptions, as well as answer questions. The robotic pharmacy system 100 may be configured to operate in a way that prevents cross-contamination between medications handled by common mechanisms, which may take the form of self-cleaning components or disposable components.

The robotic pharmacy system 100 may be a room sized secured building or container and include all necessary devices required to fill and dispense any type of prescription medication (e.g., solid drugs, liquid drugs, reconstituted liquid drugs, gels and ointments). The robotic pharmacy 100 may include a prescription filling unit 110 and a finished prescription unit 160 both disposed within an enclosure 115 defining a controlled access space 105 (e.g., secured building or container).

The prescription filling unit 110 may include a medication storage and retrieval system 120 (e.g., dispensing cabinet(s), shelving and picking system). For example, the medication storage and retrieval system 120 may be configured to store a large variety of bulk pill and tablet containers, prepackaged bottles of liquid medications, prepackaged tubes of ointments/gels (e.g., a typical assortment of bulk medications usually found in the storage areas of a manned pharmacy).

In one example, the medication storage and retrieval system 120 may be a secured cabinet or plurality of cabinets configured to securely store and dispense bulk containers and individual packages of prescription medications to the other devices within the prescription filling unit 110, as well as to receive back and store again the dispensed bulk containers once the correct amount of medications have been removed to fill a current prescription order. In this example, the dispensing cabinet(s) would not be accessible to unauthorized people who gained access into the controlled access space 105.

In another example, the medication storage and retrieval system 120 may be an open array of shelving and/or storage drawers with a picking device (e.g., robotic arm). In this example, any person who gains access to the controlled access space 105 will have access to the bulk drug containers within the medication storage and retrieval system 120. Accordingly, the controlled access space 105 may be accessible only to authorized personnel who have been verified and/or authenticated to obtain entry.

In another example, the medication storage and retrieval system 120 may include an automated pill dispensing system having multiple storage and dispensing stations, each having a storage container for storing a solid medication (e.g., pills, tablets, capsules). Here, the automated pill dispensing system may release the desired number of solid medications from one or more storage and dispensing stations into a collection device (e.g., hopper, collection container) or even into the final prescription container (e.g., vial, bottle).

The prescription filling unit 110 may include a pill counting and packaging station 130 that is configured to obtain or receive bulk solid drug (e.g., pills, tablets, capsules) containers from the medication storage and retrieval system 120, remove and verify the appropriate number of solid drugs from the bulk container, and package the removed/verified solid drugs into an appropriate container or package (e.g., bottle, vial) for dispensing to the patient. In aspects of the disclosure, the pill counting and packaging station 130 may include any of a manipulation device (e.g., robotic arm), a weighing device (e.g., scale), a counting device (e.g., optical sensor, camera, mechanical sensor) and a storage system for storing prescription containers (e.g., vials, bottles, boxes). In aspects of the disclosure, the pill counting and packaging station 130 may be configured to receive the appropriate number of solid drugs to fill a prescription directly from the medication storage and retrieval system 120, where the appropriate number of solid drugs are removed from the bulk containers by the medication storage and retrieval system 120.

For example, to fill an antibiotic pill prescription for a patient, the pill counting and packaging station 130 may receive a bulk bottle of 500 antibiotic pills from the medication storage and retrieval system 120, remove ten pills from the bulk bottle, verify there are ten pills by counting or weighing the pills, place the ten pills into a standard prescription vial and secure a child-proof top on the vial. In another example where the medication storage and retrieval system 120 has an automated pill dispensing system, the pill counting and packaging station 130 may receive the standard prescription vial already containing the ten antibiotic pills from the automated pill dispensing system and then secure a child-proof top on the vial, or receive the standard prescription vial already containing the ten antibiotic pills and a secured lid from the automated pill dispensing system. In aspects of the disclosure, the medication storage and retrieval system 120 and the pill counting and packaging station 130 may be one integral system/device.

The prescription filling unit 110 may include a liquid filling station 140 configured to obtain or receive bulk liquid containers from the medication storage and retrieval system 120, remove and verify the appropriate volume of liquid from the bulk container, and package the removed/verified volume of liquid into an appropriate container (e.g., bottle) for dispensing to the patient. In aspects of the disclosure, the liquid filling station 140 may include any of a manipulation device (e.g., robotic arm), a weighing device (e.g., scale), a volume counting device (e.g., optical sensor, camera, mechanical sensor) and a storage system for storing containers (e.g., bottles).

For example, to fill a liquid antibiotic prescription for a patient, the liquid filling station 140 may receive a bulk bottle of one liter of liquid antibiotic from the medication storage and retrieval system 120, remove 100 mL from the bulk bottle, verify there is 100 mL by weighing the removed liquid before and/or after placing the 100 mL of liquid into a standard liquid prescription bottle and secure a child-proof top on the bottle.

The prescription filling unit 110 may include a labeling and packaging station 150 configured to print and affix a label on the appropriate medication container/package received from the pill counting and packaging station 130 or the liquid filling station 140. In aspects of the disclosure, the labeling and packaging station 150 may include any of a manipulation device (e.g., robotic arm), a printer, storage for packaging (e.g., bags), labels and instruction forms, and a securing device (e.g., stapler) for sealing the packaging.

For example, in use the labeling and packaging station 150 may print a label from a roll of blank labels, remove the printed label from the roll and place the printed label around/on the prescription vial of ten antibiotic pills or the liquid prescription bottle of antibiotic liquid discussed above. The labeling and packaging station 150 may also print any number of pages of instructions and/or medication information. The printed label and/or the printed instructions may provide any desired prescription information, such as quantity, date of filling, dosage instructions, potential side effects, prescribing physician, etc., for example. Once labeled, the labeling and packaging station 150 may place the prescription vial into a prescription bag and staple the bag closed. The printed instructions may be placed inside the bag or may be stapled to the outside of the bag, for example.

The finished prescription unit 160 may include a finished prescription storage device 170 configured to receive filled prescriptions from the prescription filling unit 110 and storing them for dispensing to the patient. In aspects of the disclosure, the finished prescription storage device 170 may include any of a manipulation device (e.g., robotic arm), a scanner or imaging device, and a storage system (e.g., shelving, drawers).

For example, the finished prescription storage device 170 may receive the packaged prescription of ten antibiotic pills or 100 mL of antibiotic liquid from the prescription filling unit 110 and place the packaged prescription in a storage location. The finished prescription storage device 170 may also remove the packaged prescription from the storage location and place the packaged description in a front-end delivery space for the patient to receive.

The finished prescription unit 160 may also include a reconstitution station 180 configured to receive a filled prescription of powder or concentrated liquid in the appropriate container from the prescription filling unit 110 or retrieve the appropriate container with the powder/concentrated liquid from the storage device 170, remove the top/lid from the container, add an appropriate amount of water to the container, shake/stir the mixture in the container, secure the top/lid on the container and then provide the container for immediate dispensing to the patient. In aspects of the disclosure, the reconstitution station 180 may be included within the prescription filling unit 110, thus providing the reconstituted container to the labeling and packaging station 150 for the labeling, instruction printing and final packaging process.

The robotic pharmacy system 100 may further include an interface kiosk 190 configured to receive input from a patient and to dispense a filled prescription out to the patient. The interface kiosk 190 may be coupled to the controlled access space 105 adjacent to the finished prescription unit 160. The interface kiosk 190 may be positioned at a driving lane for drive-through access, positioned at a sidewalk for walk-up access, and positioned inside a secure lobby or airlock for access only by authorized people (e.g., authenticated customer). In aspects of the disclosure, the robotic pharmacy system 100 may include multiple interface kiosks 190, such as one drive-up kiosk 190 and one walk-up kiosk 190, for example.

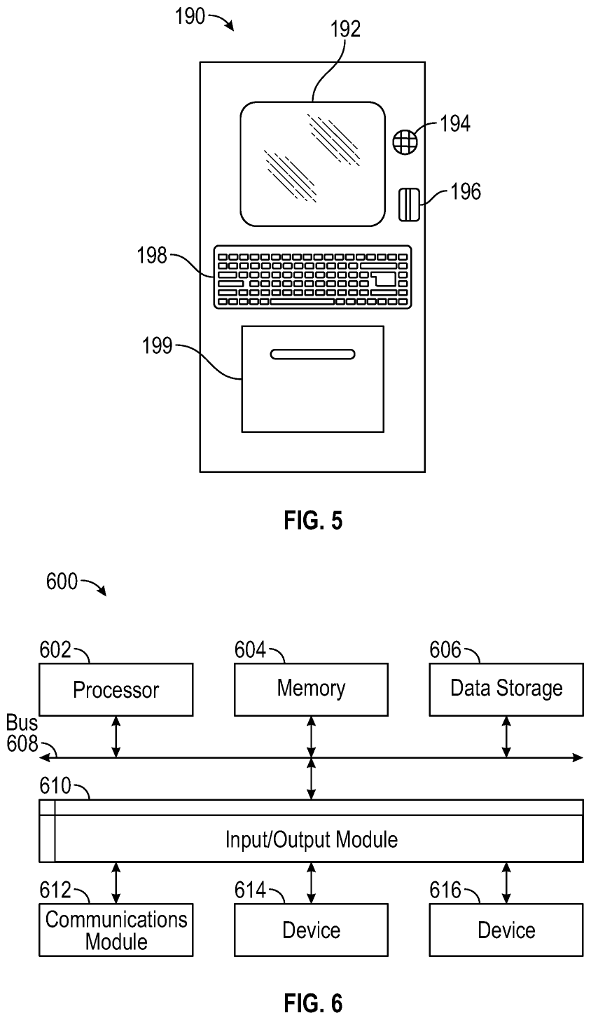

As shown in FIG. 5, the interface kiosk 190 may include a display 192 (e.g., flat panel video display), an audio device 194 (e.g., speaker, microphone), a scanning device 196 (e.g., credit card reader, ID reader, bar code scanner, QR code scanner, near field reader (NFR) scanner), an input device 198 (e.g., keyboard, touchscreen) and a delivery device 199 (e.g., bin, drawer). In aspects of the disclosure, the interface kiosk 190 may include a communication interface configured to provide an audiovisual connection to a remote pharmacist via an audiovisual device (e.g., display 192 and audio device 194).

In use, a patient may drive up or walk up to the interface kiosk 190, enter logon/authorization credentials (e.g., ID/password, QR code, prescription card) via the scanning device 196 (e.g., via a user provided medium) and/or the input device 198 (e.g., via typed or spoken user input), view their prescriptions and instructions via the display 192, enter payment via the scanning device (e.g., insert/tap credit card), and retrieve the filled prescription(s) via the delivery device 199. If the patient has any questions, the patient may communicate with a remote pharmacist via the display 192 and/or the audio device 194. For example, many jurisdictions require that a pharmacist be virtually or physically available to provide advice about prescribed medications, so the kiosk 190 may require the ability to provide a patient with virtual access to a pharmacist at the patient's request.

In aspects of the disclosure, the robotic pharmacy system 100 is completely unattended with regards to the patient experience. For example, no onsite attendance by any pharmacy staff is required during filling of a prescription and the patient's subsequent interaction with the robotic pharmacy system 100. Here, a pharmacist or staff member would only need to come to the remote site of the robotic pharmacy system 100 in order to refill/restock bulk medications, packaging, labels, etc., or to provide maintenance and repair to any of the devices and systems.

In use, the robotic pharmacy system 100 may be configured as a standalone structure connected to water and sewer lines for bringing in water and flushing out wastewater, as well as a power source (e.g., electric line from power grid). For example, the robotic pharmacy system 100 may be a small standalone building with a drive-up lane with a drive-up kiosk 190 and/or a walk-up lane with a walk-up kiosk 190. The building may also include communications connections (e.g., telephone, internet) as well as heating, ventilation and air conditioning (HVAC) systems (e.g., furnace and air conditioner). The robotic pharmacy system 100 may be configured with an emergency power source (e.g., a generator).

In aspects of the disclosure, the robotic pharmacy system 100 may be attached to or part of another building structure. For example, the robotic pharmacy system 100 may be a portion of a medical building (e.g., doctor's office building, clinic) or of a retail establishment (e.g., drug store). Here, the robotic pharmacy system 100 may be connected to the utilities (e.g., water, sewer, gas, electric, HVAC, internet) of the larger building structure. The robotic pharmacy system 100 may have multiple kiosks 190, such as an outdoor drive-up kiosk, an outdoor walk-up kiosk and/or an indoor kiosk (e.g., inside a small lobby or airlock for weather proofing and security). As another example, the robotic pharmacy system 100 may be attached to an urgent care center or an emergency room that permits patients seen in such centers to receive prescriptions issued during their visit to the urgent care center or emergency room.

In aspects of the disclosure, the robotic pharmacy system 100 may be a movable structure with wheels (e.g., trailer, mobile home) or a movable structure without wheels (e.g., container, storage pod). For example, the robotic pharmacy system 100 may be a mobile pharmacy that can be driven from location to location to serve a wide range of rural or underserved communities. Here, the mobile robotic pharmacy system 100 may be located in a different town each day of the week, for example. As another example, the robotic pharmacy system 100 may be a customized shipping container or storage pod that is dropped into place for extended periods of time.

In aspects of the disclosure, any of the robotic pharmacy systems 100 discussed above may have their own utility systems (e.g., generator, water tank, waste tank, communications server, HVAC systems).

In aspects of the disclosure, the robotic pharmacy system 100 may include environmental monitoring, controls and alarms to ensure that the proper storage conditions are maintained for the medications.

In aspects of the disclosure, the robotic pharmacy system 100 may include security systems that would call local police if there were attempted or actual unauthorized entry into the device for the purpose of stealing medications.

In aspects of the disclosure, the robotic pharmacy system 100 may include further secured storage for controlled substances, which legally requires more rigorous protection against theft as well as more rigorous accounting for medications.

In aspects of the disclosure, the robotic pharmacy system 100 may be configured to maintain supplies of commonly used non-prescription medications that the patient could purchase along with, or instead of prescription medications. While some jurisdictions require verification of the buyer's identity for purchases of some non-prescription medications (e.g., pseudoephedrine), in the case where only applicable non-prescription medication were purchased, the robotic pharmacy system 100 would not need to verify patient identity.

In aspects of the disclosure, the robotic pharmacy system 100 may include one or more camera systems through which a remote pharmacist may double check/verify filled prescriptions. For example, the robotic pharmacy system 100 may provide a still or video camera view of the number of pills, volume of liquid, or number of tubes of medication provided by the medication storage and retrieval system 120 to the pill counting and packaging station 130, the liquid filling station 140 and/or the labeling and packaging station 150. Here, a remote pharmacist may view the camera images to verify that the correct amount of medication has been provided.

In aspects of the disclosure, the robotic pharmacy system 100 may include a camera for viewing and verifying the printed label and/or printed instructions. In aspects of the disclosure, the robotic pharmacy system 100 may include a camera for viewing and verifying the final prescription package before it is dispensed to the patient. Such cameras may provide for the remote pharmacist to comply with all health and regulatory rules required in order to dispense medications to the patient.

In aspects of the disclosure, the robotic pharmacy system 100 may include a refrigeration unit for keeping certain medications at a required temperature. In aspects of the disclosure, the robotic pharmacy system 100 may include a humidifier unit and/or a dehumidifier unit for keeping certain medications in a required humidity range.

FIG. 6 is a block diagram illustrating an example computer system 600 with which the robotic pharmacy system 100 of FIGS. 4 and 5 can be implemented. In certain aspects, the computer system 600 may be implemented using hardware or a combination of software and hardware, either in a dedicated server, or integrated into another entity, or distributed across multiple entities.

Computer system 600 (e.g., an automated medication dispensing system) includes a bus 608 or other communication mechanism for communicating information, and a processor 602 coupled with bus 608 for processing information. By way of example, the computer system 600 may be implemented with one or more processors 602. Processor 602 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 600 can include, in addition to hardware, code that creates an execution environment for the computer program in question, e.g., code that constitutes processor firmware, a protocol stack, a database management system, an operating system, or a combination of one or more of them stored in an included memory 604, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, cloud storage, or any other suitable storage device, coupled to bus 608 for storing information and instructions to be executed by processor 602. The processor 602 and the memory 604 can be supplemented by, or incorporated in, special purpose logic circuitry.

The instructions may be stored in the memory 604 and implemented in one or more computer program products, i.e., one or more modules of computer program instructions encoded on a computer readable medium for execution by, or to control the operation of, the computer system 600, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, embeddable languages, and xml-based languages. Memory 604 may also be used for storing temporary variable or other intermediate information during execution of instructions to be executed by processor 602.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 600 further includes a data storage device 606 such as a magnetic disk or optical disk, coupled to bus 608 for storing information and instructions. Computer system 600 may be coupled via input/output module 610 to various devices. The input/output module 610 can be any input/output module. Example input/output modules 610 include data ports such as USB ports. The input/output module 610 is configured to connect to a communications module 612. Example communications modules 612 include networking interface cards, such as Ethernet cards and modems. In certain aspects, the input/output module 610 is configured to connect to a plurality of devices, such as an input device 614 and/or an output device 616. Example input devices 614 include a keyboard and a pointing device, e.g., a mouse or a trackball, by which a user can provide input to the computer system 600. Other kinds of input devices 614 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Example output devices 616 include display devices, such as a LED (light emitting diode), CRT (cathode ray tube), or LCD (liquid crystal display) screen, for displaying information to the user.

According to one aspect of the present disclosure, the inventory management system can be implemented using a computer system 600 in response to processor 602 executing one or more sequences of one or more instructions contained in memory 604. Such instructions may be read into memory 604 from another machine-readable medium, such as data storage device 606. Execution of the sequences of instructions contained in main memory 604 causes processor 602 to perform the process steps described herein. One or more processors in a multi-processing arrangement may also be employed to execute the sequences of instructions contained in memory 604. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the subject matter described in this specification), or any combination of one or more such back end, middleware, or front end components. The components of the system can be interconnected by any form or medium of digital data communication (e.g., a communication network). The communication network can include, for example, any one or more of a personal area network (PAN), a local area network (LAN), a campus area network (CAN), a metropolitan area network (MAN), a wide area network (WAN), a broadband network (BBN), the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computing system 600 can include clients and servers. A client and server are generally remote from each other and typically interact through a communication network. The relationship of client and server arises by virtue of computer programs running on the respective computers and having a client-server relationship to each other. Computer system 600 can be, for example, and without limitation, a desktop computer, laptop computer, or tablet computer. Computer system 600 can also be embedded in another device, for example, and without limitation, a mobile telephone, a personal digital assistant (PDA), a mobile audio player, a Global Positioning System (GPS) receiver, a video game console, and/or a television set top box.

Computing system 600 can include software that actually operates the individual components as firmware within those devices, a coordinating software system at the overall robotic system level that can direct tasks to the various robotic components as required, a distinct and separate software system for the kiosk, and a separate software system that can perform workflow management for the local robotic system. That workflow system can in turn be connected to an enterprise software system (e.g., in the cloud) that can govern a variety of these robotic systems, direct prescriptions to the appropriate robotic system, manage tasking for refilling and servicing these robotic systems, and manage communications between the kiosk and remote pharmacists providing patient counseling and instruction.

The term “machine-readable storage medium” or “computer readable medium” as used herein refers to any medium or media that participates in providing instructions or data to processor 602 for execution. Such a medium may take many forms, including, but not limited to, non-volatile media, volatile media, and transmission media. Non-volatile media include, for example, optical disks, magnetic disks, or flash memory, such as data storage device 606. Volatile media include dynamic memory, such as memory 604. Transmission media include coaxial cables, copper wire, and fiber optics, including the wires that comprise bus 608. Common forms of computer-readable media or machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

One or more embodiments include an unattended robotic pharmacy system, comprising: an enclosure defining a controlled access space; a prescription filling unit disposed within the controlled access space, the prescription filling unit configured to package and label a patient prescription of medication obtained from medication storage within the prescription filling unit; a finished prescription unit disposed within the controlled access space and coupled to the prescription filling unit, the finished prescription unit configured to store the packaged and labeled patient prescription obtained from the prescription filling unit; and an interface kiosk coupled to the enclosure and disposed adjacent the finished prescription unit, the interface kiosk configured to receive input from a user and to dispense a finished prescription obtained from the finished prescription unit to the user.

In aspects of the disclosure, the prescription filling unit comprises a manipulation device. In aspects of the disclosure, the prescription filling unit comprises a medication storage and retrieval system configured to store bulk quantities of a plurality of medications within a plurality of containers. In aspects of the disclosure, the plurality of containers includes one or more of bulk solid drug portion containers, bulk liquid containers, bulk containers of individual ointment tubes and bulk powder containers. In aspects of the disclosure, the medication storage and retrieval system comprises a manipulation device. In aspects of the disclosure, the prescription filling unit comprises a pill counting and packaging station configured to: count and confirm an appropriate number of solid drug portions to fill the patient prescription; and package the appropriate number of solid drug portions into a prescription container. In aspects of the disclosure, the pill counting and packaging station comprises a manipulation device.

In aspects of the disclosure, the prescription filling unit comprises a liquid filling station configured to: measure and confirm an appropriate volume of liquid medication to fill the patient prescription; and package the liquid medication into a prescription container. In aspects of the disclosure, the liquid filling station comprises a manipulation device. In aspects of the disclosure, the prescription filling unit comprises a labeling and packaging station configured to print and affix a label to a filled prescription container. In aspects of the disclosure, the labeling and packaging station is configured to package the filled prescription container in a final prescription package. In aspects of the disclosure, the labeling and packaging station is configured to print and affix prescription instructions to the final prescription package. In aspects of the disclosure, the labeling and packaging station comprises a manipulation device.

In aspects of the disclosure, the finished prescription unit comprises: a storage system configured to receive and hold a plurality of final prescription packages received from the prescription filling unit; and one of a scanner and an imaging device. In aspects of the disclosure, the finished prescription unit comprises a manipulation device. In aspects of the disclosure, the finished prescription unit comprises a reconstitution station configured to add water to a prescription container containing one of a medication powder and a concentrated medication liquid.

In aspects of the disclosure, the interface kiosk comprises: a display configured to provide visual information to the user; an audio device configured to one of provide audio information to the user and receive audio input from the user; a communication interface configured to provide an audiovisual connection to a remote pharmacist via an audiovisual device; an input device configured to receive information entered by the user; a scanning device configured to read information from a user provided medium; and a delivery device configured to provide user access to the finished prescription. In aspects of the disclosure, the interface kiosk is configured as one of a drive-up kiosk and a walk-up kiosk. In aspects of the disclosure, the unattended robotic pharmacy system comprises a utility connection to one or more of a water line, a sewer line, an electric power line, a communication line and a heating/cooling duct. In aspects of the disclosure, the unattended robotic pharmacy system comprises one or more of a water storage container, a waste storage container, a power generator, a communication server and a heating/cooling unit.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

As used herein, the term “message” encompasses a wide variety of formats for communicating (e.g., transmitting or receiving) information. A message may include a machine readable aggregation of information such as an XML document, fixed field message, comma separated message, or the like. A message may, in some implementations, include a signal utilized to transmit one or more representations of the information. While recited in the singular, it will be understood that a message may be composed, transmitted, stored, received, etc. in multiple parts.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

In any embodiment, data can be forwarded to a “remote” device or location,” where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

Some embodiments include implementation on a single computer, or across a network of computers, or across networks of networks of computers, for example, across a network cloud, across a local area network, on hand-held computer devices, etc. The computers may be physical machines or virtual machines hosted by other computers. In certain embodiments, one or more of the steps described herein are implemented on a computer program(s). Such computer programs execute one or more of the steps described herein. In some embodiments, implementations of the subject method include various data structures, categories, and modifiers described herein, encoded on computer-readable medium(s) and transmissible over communications network(s).

Software, web, internet, cloud, or other storage and computer network implementations of the present invention could be accomplished with standardized programming techniques specifically adapted to cause one or more device to perform the various assigning, calculating, identifying, scoring, accessing, generating or discarding steps described.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

1. An unattended robotic pharmacy system comprising:
an enclosure defining a controlled access space;
a prescription filling unit disposed within the controlled access space, the prescription filling unit configured to package and label a patient prescription of medication obtained from medication storage within the prescription filling unit;
a finished prescription unit disposed within the controlled access space and coupled to the prescription filling unit, the finished prescription unit configured to store the packaged and labeled patient prescription obtained from the prescription filling unit;
an interface kiosk coupled to the enclosure and disposed adjacent the finished prescription unit, the interface kiosk configured to receive input from a user and to dispense a finished prescription obtained from the finished prescription unit to the user;
a first utility connection to a water supply to deliver water inside the enclosure;
a second utility connection to a sewer line to deliver waste out of the enclosure; and
a third utility connection to an electric power line to deliver power into the enclosure to at least one of the prescription filling unit, the finished prescription unit, or the interface kiosk.

2. The unattended robotic pharmacy system of claim 1, wherein the prescription filling unit comprises a manipulation device.

3. The unattended robotic pharmacy system of claim 1, wherein the prescription filling unit comprises a medication storage and retrieval system configured to store bulk quantities of a plurality of medications within a plurality of containers.

4. The unattended robotic pharmacy system of claim 3, wherein the plurality of containers includes one or more of bulk solid drug portion containers, bulk liquid containers, bulk containers of individual ointment tubes and bulk powder containers.

5. The unattended robotic pharmacy system of claim 3, wherein the medication storage and retrieval system comprises a manipulation device.

6. The unattended robotic pharmacy system of claim 1, wherein the prescription filling unit comprises a pill counting and packaging station configured to:
count and confirm an appropriate number of solid drug portions to fill the patient prescription; and
package the appropriate number of solid drug portions into a prescription container.

7. The unattended robotic pharmacy system of claim 6, wherein the pill counting and packaging station comprises a manipulation device.

8. The unattended robotic pharmacy system of claim 1, wherein the prescription filling unit comprises a liquid filling station configured to:
receive water via the first utility connection;
measure and confirm an appropriate volume of liquid medication to fill the patient prescription;
package the liquid medication into a prescription container; and
transfer waste from filling the patient prescription to the second utility connection.

9. The unattended robotic pharmacy system of claim 8, wherein the liquid filling station comprises a manipulation device.

10. The unattended robotic pharmacy system of claim 1, wherein the prescription filling unit comprises a labeling and packaging station configured to print and affix a label to a filled prescription container.

11. The unattended robotic pharmacy system of claim 10, wherein the labeling and packaging station is configured to package the filled prescription container in a final prescription package.

12. The unattended robotic pharmacy system of claim 11, wherein the labeling and packaging station is configured to print and affix prescription instructions to the final prescription package.

13. The unattended robotic pharmacy system of claim 10, wherein the labeling and packaging station comprises a manipulation device.

14. The unattended robotic pharmacy system of claim 1, wherein the finished prescription unit comprises:
a storage system configured to receive and hold a plurality of final prescription packages received from the prescription filling unit; and
one of a scanner and an imaging device.

15. The unattended robotic pharmacy system of claim 14, wherein the finished prescription unit comprises a manipulation device.

16. The unattended robotic pharmacy system of claim 14, wherein the finished prescription unit comprises a reconstitution station configured to add water to a prescription container containing one of a medication powder and a concentrated medication liquid.

17. The unattended robotic pharmacy system of claim 1, wherein the interface kiosk comprises:
a display configured to provide visual information to the user;
an audio device configured to one of provide audio information to the user and receive audio input from the user;
a communication interface configured to provide an audiovisual connection to a remote pharmacist via an audiovisual device;
an input device configured to receive information entered by the user;
a scanning device configured to read information from a user provided medium; and
a delivery device configured to provide user access to the finished prescription.

18. The unattended robotic pharmacy system of claim 17, wherein the interface kiosk is configured as one of a drive-up kiosk and a walk-up kiosk.

19. The unattended robotic pharmacy system of claim 1, comprising a utility connection to one or more of a communication line and a heating/cooling duct.

20. An unattended robotic pharmacy system, comprising:
an enclosure defining a controlled access space;
a prescription filling unit disposed within the controlled access space, the prescription filling unit configured to package and label a patient prescription of medication obtained from medication storage within the prescription filling unit;
a finished prescription unit disposed within the controlled access space and coupled to the prescription filling unit, the finished prescription unit configured to store the packaged and labeled patient prescription obtained from the prescription filling unit;
an interface kiosk coupled to the enclosure and disposed adjacent the finished prescription unit, the interface kiosk configured to receive input from a user and to dispense a finished prescription obtained from the finished prescription unit to the user;
a water storage container;
a waste storage container;
a power generator;
a communication server; and
a heating/cooling unit.
